(19) United States
(12) Patent Application Publication
(10) Pub. No.: US 2015/0011542 A1
(43) Pub. Date: Jan. 8, 2015

(54) Methods of Treating Adverse Intestinal Effects of Non-Steroidal Anti-Inflammatory Drugs

(71) Applicant: University of Connecticut, Farmington, CT (US)

(72) Inventor: Urs A. Boelsterli, Ellington, CT (US)

(21) Appl. No.: 14/370,980

(22) PCT Filed: Jan. 11, 2013
(86) PCT No.: PCT/US2013/021157

§ 371 Date: Jul. 8, 2014

Related U.S. Application Data
(60) Provisional application No. 61/585,946, filed on Jan. 12, 2012.

(51) Int. Cl. A61K 31/5377; A61K 45/06; A61K 31/4704; A61K 31/519; A61K 31/496

(52) U.S. Cl. CPC A61K 31/5377; A61K 31/519; A61K 31/496; A61K 31/4704; A61K 45/06
USPC 514/232.8; 514/252.16; 514/252.17; 514/254.11; 514/312

(57) ABSTRACT

Methods of treating adverse intestinal effects of a non-steroidal anti-inflammatory drugs (NSAID) in a subject by administering to the subject a therapeutically effective amount of a selective bacterial beta-glucuronidase inhibitor. The adverse intestinal effects to be treated include the formation or growth of an intestinal ulcer, increased intestinal permeability, the loss of intestinal villi, bleeding of the intestinal mucosa, and an increased intestinal inflammatory response. The methods are useful for treating the adverse effects of any NSAID such as propionic acid derivatives, carboxylic acid derivatives, enolic acid derivatives, fenamic acid derivatives, sulphonanilidies, and selective COX-2 inhibitors. The bacterial beta-glucuronidase inhibitors are selective for the inhibition of bacterial beta-glucuronidase enzymes and do not inhibit mammalian beta-glucuronidase.

### STATEMENT AS TO RIGHTS TO INVENTIONS MADE UNDER FEDERALLY SPONSORED RESEARCH AND DEVELOPMENT

This invention was made with government support under the National Institutes of Health Grant CA98468. The government has certain rights in this invention.

### FIELD OF THE INVENTION

The present application relates to methods for treating the adverse gastrointestinal effects caused by nonsteroidal anti-inflammatory drugs (NSAIDs) using selective bacterial β-glucuronidase inhibitors.

### BACKGROUND OF THE INVENTION

Nonsteroidal anti-inflammatory drugs (NSAIDs) are drugs with analgesic, antipyretic and anti-inflammatory effects that extensively used worldwide. Aspirin, ibuprofen and naproxen are examples of NSAIDs available without a prescription. NSAIDs are classified based on their chemical structure or mechanism of action. Several NSAID classifications include: salicylates, p-amino phenol derivative, propionic acid derivatives, carboxylic acid derivatives, enolic acid derivatives, fenamic acid derivatives, and sulphonanilides. Most NSAIDs act as nonselective inhibitors of the enzyme cyclooxygenase (COX) and inhibit both COX-1 and COX-2. The COX protein catalyzes the formation of prostaglandins and thromboxane from arachidonic acid. Prostaglandins effectuate fever and inflammation in individuals, and accordingly, NSAID administration to an individual results in the reduction of fever and inflammation.

Gastrointestinal (GI) injury is one of the major adverse effects associated with NSAIDs. This iatrogenic disease is manifested as ulceration and bleeding of the mucosa, inflammation, and even perforation (Allison et al., New Engl. J. Med, 327:749-754 (1992); Bjarnason et al., Gastroenterology, 104:1832-1847 (1993); Wolfe et al., New Engl. J. Med., 340:1888-1899 (1999)). With the advent of novel diagnostic tools including video capsule endoscopy, it has become increasingly clear in recent years that not only the stomach, but also the small intestine is a major target of NSAID-associated toxicity (Davies et al., J. Pharmacy Pharm. Sci., 3:137-155 (2000); Koga et al., Gastroenterol. Endosc., 50:189-198 (2008); Scarpignato and Hunt, Gastroenterol. Clin. N. Am., 39:433-464 (2010)). Indeed, approximately two-thirds of both long-term (greater than 3 months) and short-term (approximately one week) NSAID users exhibited mild or more severe forms of drug-induced lesions in the small intestine (Fortun and Hawkey, Curr. Opin. Gastroenterol., 23:134-141 (2007); Maiden, J. Gastroenterol., 44:64-71 (2009)). In addition, many unexplained GI lesions in “control subjects” were found to be attributable to non-prescription use of NSAIDs (Sidhu et al., Clin. Gastoenterol. Hepatol., 8:992-995 (2010)). Despite this high incidence of the disease, there are currently no approved therapies to prevent or treat NSAID enteropathy.

Part of the reason for a lack of therapies is an incomplete understanding of the underlying mechanisms of NSAID enteropathy (Whittle, Eur. J. Pharmacol., 500:427-439 (2004)). The mode of toxicity to the small intestinal mucosa is clearly distinct from that involved in the precipitation of gastric lesions induced by NSAIDs. In fact, co-administration of a proton pump inhibitor (commonly used to provide gastroprotection against NSAIDs) has been shown to have little effect, and even to exacerbate small intestinal ulceration in both patients and rodent models (Goldstein et al., Clin. Gastoenterol. Hepatol., 3:133-141 (2005); Wallace et al., Gastroenterology, 141:1314-1322 (2011)).

The inhibition of COX-1 and/or COX-2 is one factor that may contribute to the NSAID toxicity (Sigthorsson et al., Gastroenterology, 122:1913-1923 (2002); Tanaka et al., J. Pharmacol. Exp. Ther., 300:754-761 (2002); Hotz-Behofsits et al., Scand. J. Gastroenterol., 45:822-827 (2010)). It has been shown that reduced inhibition of COX-1, or targeted inhibition of COX-2, can reduce the gastrointestinal side effects of NSAIDs. However, selective COX-2 inhibitors also have cardiovascular side effects that have resulted in their withdrawal from the market. Furthermore, the pathogenesis of mucosal damage and subsequent ulceration is clearly a multistep process such that its treatment or prevention requires the targeting of more than selective alterations in the inhibition of COX-1 and/or COX-2.

In an effort to better understand the underlying mechanisms of NSAID gastrointestinal effects, the in vivo metabolism of many NSAIDS has been investigated, including the carboxylic acid derivative, diclofenac (DCF). It has been found that a portion of the hepatic diclofenac pool is conjugated with glucuronic acid to form a water-soluble 1-β-O-acyl glucuronide. This acyl glucuronide (AG) is readily excreted across the hepatocanalicular membrane via Mrp2 into the biliary tree (Seitz and Boelsterli, Gastroenterology, 115:1476-1482 (1998)) and delivered to more distal sites, i.e., the jejunum and ileum (Boelsterli and Ramirez-Alcantara, Curr. Drug Metab., 12:245 (2011)). During this transport, a portion of the AG is converted to iso-glucuronides by spontaneous acyl migration of the aglycone along the sugar ring (Dickinson and King, Life Sci., 70:25-36 (2001)). Diclofenac AG (but not the iso-glucuronides) can be cleaved by bacterial β-glucuronidase in the lumen of the small bowel. The released DCF is then taken up by enterocytes (the intestinal absorptive cells) and undergoes enterohepatic circulation.

Among the network of mechanisms involved in the initial injury to enterocytes, both ER stress and mitochondrial stress have been implicated as critical mediators of cell death (Somasundaram et al., Gut, 41:344-353 (1997); Somasundaram et al., Aliment. Pharmacol. Ther., 14:639-650 (2000); Tsutsumi et al., Cell Death Different, 11:1009-1016 (2004); Tanaka et al., J. Biol. Chem., 280:31059-31067 (2005); Ramirez-Alcantara et al., Am. J. Physiol. (Gastointest. Liver Physiol.), 297:G990-G998 (2009); LoGuidice et al., Toxicol. Sci., 118:276-285 (2010); Watanabe et al., J. Clin. Biochem., 48:117-121 (2011)). Furthermore, and importantly, after this “first hit”, a subsequent “second hit” and inflammatory response is triggered by the increased permeability of the gut mucosa. This involves intestinal bacterial lipopolysaccharide (LPS)-mediated activation of Toll-like receptor 4 (TLR4) on macrophages, leading to tumor necrosis factor-mediated cell injury and secondary activation of the innate immune system and recruitment of inflammatory cells to the site of injury (Watanabe et al., Gut, 57:181-187 (2008)).

Previous studies have aimed at targeting one or more of these network pathways in an attempt to develop cytoprotective strategies against NSAID enteropathy (Watanabe et al., Gut, 57:181-187 (2008); Ramirez-Alcantara et al., Am. J. Physiol. (Gastrointest. Liver Physiol.), 297:G990-G998 (2009); LoGuidice et al., Toxicol. Sci., 118:276-285 (2010); Yamada et al., Gastroenterol. Hepatol., 26:398-404 (2011)). However, none of these studies have resulted in a successful treatment or method for preventing NSAID enteropathy. Studies performed many decades ago did show that the NSAID indomethacin administered to either germ-free rats or to rats treated with antibiotics reduced the extent of intestinal injury (Kent et al., Am. J. Pathol., 54:237-248 (1969); Robert and Asano, Prostaglandins, 14:333-341 (1977)). The elimination of intestinal bacteria did abrogate the source of LPS and, hence, reduce the inflammatory response in the gut. However, this procedure also decreases the ability of the small intestine to hydrolyze drug glucuronides. Because a normal gut flora is important for maintaining a normal health status, such approaches are less suitable for therapeutic applications.

Accordingly, there is a considerable need for safe and effective methods for reducing the adverse effects of NSAIDs in the intestine of a subject or individual.

### SUMMARY OF THE INVENTION

The methods provided herein answer the need for safe and effective methods for treating the adverse effects of NSAIDs in the intestine of a subject or individual. Methods of treating one or more adverse intestinal effects of an NSAID in a subject by administering to the subject a therapeutically effective amount of a selective bacterial β-glucuronidase inhibitor are described herein. The adverse intestinal effects to be treated include the formation or growth of an intestinal ulcer, increased intestinal permeability, the loss of intestinal villi, bleeding of the intestinal mucosa, and an increased intestinal inflammatory response.

The methods described herein are useful for treating the adverse effects of any NSAID such as, but not limited to, propionic acid derivatives, carboxylic acid derivatives, enolic acid derivatives, fenamic acid derivatives, sulphonanilides, and selective COX-2 inhibitors. In some embodiments, the NSAID is a carboxylic acid derivative such as, but not limited to, indomethacin, sulindac, etodolac, ketorolac, and diclofenac. In one embodiment, the NSAID is diclofenac. In other embodiments, the NSAID is a propionic acid derivative such as ketoprofen.

The bacterial β-glucuronidase inhibitors used in the present methods are selective for the inhibition of bacterial β-glucuronidase enzymes and do not inhibit mammalian β-glucuronidase. The present methods provide for the first time a method of using these selective bacterial β-glucuronidase inhibitors for the treatment of one or more adverse intestinal effects of an NSAID. The chemical formulas of selective bacterial β-glucuronidase inhibitors are described below.

Due to the copious worldwide usage of NSAIDs, providing a safe and effective treatment for the gastrointestinal adverse effects of NSAIDs is an important scientific advance. Furthermore, in view of the increased life expectancy in developed countries and the associated shifts in the demographic patterns, there will undoubtedly be an increased future demand for anti-rheumatic drugs such as NSAIDs. Due to the known risks of the selective COX-2 inhibitors for developing cardiovascular complications, many health professionals will increasingly prescribe the more traditional NSAIDs (non-selective COX inhibitors) such as the carboxylic acid NSAIDs. Accordingly, the present methods provide an important advance in the treatment of the adverse intestinal effects of NSAIDs that will likely be the subject of increased use over time.

### BRIEF DESCRIPTION OF THE DRAWINGS

FIG. 1 illustrates that diclofenac-1-β-O acyl glucuronide is a substrate for E. coli β-glucuronidase and that Inh-1 inhibits this enzymatic hydrolysis reaction. (A) Chemical structure of Inh-1 and conjugation-deconjugation cycling of diclofenac (abbreviated DCF) and its inhibition by Inh-1 UGT2B7, uridine diphosphate glucuronosyl transferase 2B7; UDPGA, uridine diphosphate glucuronic acid. (B) In vitro studies with purified E. coli β-glucuronidase and DCF-AG (4 mM) were performed and enzymatic generation of the product, free DCF, was assessed by HPLC analysis. Inh-1 caused a concentration-dependent inhibition of product formation. AUC, area under the plasma concentration versus time curve.

FIG. 2 illustrates a macroscopic and histopathologic assessment of mucosal damage in mouse jejunum 18 hours after diclofenac administration with and without pretreatment with Inh-1. (A) Macroscopic view of the luminal side of the mouse jejunum 18 hours after DCF administration with and without pretreatment with Inh-1 ; note the large ulcers (arrow) in DCF-treated mice and the absence of any large ulceration in the Inh-1/DCF co-treated mice (NBT staining; 10× magnification). (B) Representative histopathological sections of the mouse jejunum; note the ulcer in the DCF-treated mouse involving the entire mucosa and characterized by an inflammatory response (arrow). These lesions are not present in Inh-1/DCF co-treated mice (H&E; 40× magnification).

FIG. 3 illustrates protection from diclofenac-induced small intestinal ulceration by Inh-1 pretreatment. Male C57BL/6 mice were administered a single intraperitoneal dose of DCF (60 mg/kg) or vehicle (10% Solutol HS-15) following pre-treatment with Inh-1 (10 μg/mouse, po, b.i.d.) or vehicle (0.5% methyl cellulose) for two consecutive days. (A)

Average ulcer area was determined by morphometric analysis of photomicrographs using ImageJ software. The quartile numbers (Q1-4) are assigned to four small intestinal segments of equal length from the gastroduodenal junction to the ileocecal junction. Data are mean±SD (n=7 mice per group). *, P≦0.05 versus DCF alone in the respective quartile. (B) Average large ulcer number (≧score 2). Data are mean±SD (n=7). *, P≦0.05 versus DCF alone in the respective quartile. (C) Serum FITC-dextran levels as an indication of intestinal permeability. FITC-dextran was orally administered (400 mg/kg) 15 hours post DCF, and blood was collected three hours later. Data are mean±SD (n=5). *, P≦0.05 versus vehicle control; #, versus DCF alone. (D) Serum alkaline phosphatase activity 18 hours post-DCF. Data are mean±SD (n=5). *, P≦0.05 versus vehicle control; #, versus DCF alone.

FIG. 4 illustrates the effect of Inh-1 on the pharmacokinetic profile of DCF. Male C57BL/6 mice were administered a single dose of DCF (60 mg/kg, intraperitoneal) with or without pretreatment with Inh-1 (10 μg per mouse, b.i.d., × two days). Serial blood sampling was performed at the indicated time points. Diclofenac plasma concentrations were determined by LC-MS/MS as described in the examples below. Data points are mean±SD (n=three mice per time point; maximal three time points per mouse).

### DETAILED DESCRIPTION OF THE INVENTION

### I. Introduction

Safe and effective methods for reducing the adverse effects of NSAIDs in the intestine of a subject are provided herein. In particular, the methods of reducing or preventing an adverse intestinal effect of an NSAID in an individual by administering to the subject a therapeutically effective amount of a selective bacterial β-glucuronidase inhibitor are described. Selective bacterial β-glucuronidase inhibitors for use in the present methods include, but are not limited to, those described in Wallace et al., Science, 330:831-835 (2010) as affording protection against intestinal toxicity associated with the anti-cancer drug CPT-11 (irinotecan). The inhibitors are selective in that they inhibit bacterial β-glucuronidase, but do not inhibit mammalian β-glucuronidase.

It is a surprising discovery of the present methods that selective bacterial β-glucuronidase inhibitors reduce the adverse intestinal effects of NSAIDs. Gastrointestinal injury is one of the major adverse effects associated with NSAIDs and recent advances in endoscopy, such as video capsule endoscopy, have made it increasingly clear that the small intestine is a major target of NSAID-associated toxicity. This application describes for the first time the treatment and/or prevention of NSAID adverse effects, such as intestinal ulcers attributable to NSAIDs, using a selective bacterial β-glucuronidase inhibitor. A reduction of intestinal permeability associated with NSAIDs is also described for the first time and can be achieved using the methods provided herein.

In certain embodiments, the selective bacterial β-glucuronidase inhibitor is a compound having one of chemical formulas 1-9, as described in more detail below. It is a further surprising discovery that the compound of chemical formula 1 (referred to herein as Inh-1) is a potent inhibitor of the deconjugation of the conjugate of a carboxylic acid NSAID, such as diclofenac (or DCF), with glucuronic acid. A portion of the carboxylic acid-containing NSAIDs are conjugated with glucuronic acid in vivo and form acyl glucuronides. Although not wanting to be bound by this, it is believed that inhibition of carboxylic acid

NSAID/glucuronic acid deconjugation in vivo results in a reduced exposure of the intestinal mucosa to the NSAID and thereby reduces NSAID toxicity. Propionic acid NSAIDs, enolic acid NSAIDs, fenamic acid NSAIDs, sulphonanilide NSAIDs and selective COX-2 inhibitor NSAIDs also form glucuronides. Accordingly, the method described herein can be used to inhibit deconjugation of glucuronides derived from propionic acid NSAIDs, enolic acid NSAIDs, fenamic acid NSAIDs, sulphonanilide NSAIDs, or selective COX-2 inhibitor NSAIDs and/or inhibit the adverse intestinal effects associated with propionic acid NSAIDs, enolic acid NSAIDs, fenamic acid NSAIDs, sulphonanilide NSAIDs, or selective COX-2 inhibitor NSAIDs.

### II. Definitions

As used herein, the following terms have the meanings ascribed to them unless specified otherwise.

The term “active derivative” and the like means a modified β-glucuronidase inhibiting compound that retains an ability to selectively inhibit bacterial β-glucuronidases. For example, the active derivative can be capable of inhibiting β-glucuronidase reactivation of diclofenac-AG to diclofenac, but generally not capable of killing bacteria that inhabit the gastrointestinal tract of an individual or subject or of inhibiting mammalian β-glucuronidases. The active derivative can alternatively or also be capable of inhibiting bacterial, but not mammalian, β-glucuronidases from hydrolyzing glucuronides. One of skill in the art is familiar with assays for testing the ability of an active derivative compound for selectively inhibiting β-glucuronidases that do not generally kill the normal gastrointestinal flora.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “adverse intestinal effect” refers herein to the formation or growth of an intestinal ulcer, increased intestinal permeability, the loss of intestinal villi, intestinal bleeding of the mucosa, or an increased intestinal inflammatory response. The term “enteropathy” is used herein to refer to one or more adverse intestinal effects. One of skill in the art is familiar with assays for identifying intestinal ulcers or the loss of intestinal villi, including but not limited to, macroscopic and histopathologic assays. Increased intestinal permeability can be measured as described in Example II herein or any other appropriate method known to those of skill in the art. An intestinal inflammatory response can be indicated by any method known to those of skill in the art including, but not limited to, macroscopic and histopathologic assays Enteropathy is traditionally indicated by detecting a decreased serum alkaline phosphatase level, however, any additional methods known to those of skill in the art can be employed to indicate enteropathy.

The term “62 -glucuronidase” and the like means an enzyme capable of hydrolyzing (β-glucuronides, but not α-glucuronides or β-glucosides. (Basinska & Florianczyk, Ann. Univ. Mariae Curie Sklodowska Med., 58:386-389 (2003); Miles et al., J. Biol. Chem., 217:921-930 (1955)). The term “β-glucuronidase” includes, but is not limited to, β-D-glucuronidases such as Escherichia coli β-D-glucuronidase, and β-glucuronidases derived from bacteria including Enterobacteria, Clostridia, and Bacteroides.

The term “glucuronide” and the like means a substance produced by linking glucuronic acid to another substance via a glycosidic bond. Examples of glucuronides of interest herein include NSAID glucuronides such as carboxylic acid NSAID glucuronides, propionic acid NSAID glucuronides, enolic acid NSAID glucuronides, fenamic acid NSAID glucuronides, sulphonanilide NSAID glucuronides, and selective COX-2 inhibitor NSAID glucuronides. The term “glucuronides” further includes glucuronides formed from NSAID metabolites.

The term “nonsteroidal anti-inflammatory drug” or “NSAID” and the like refer to drugs with analgesic and antipyretic effects and which have, in higher doses, anti-inflammatory effects. NSAIDs are divided into the following types or categories: salicylates, p-amino phenol derivatives, propionic acid derivatives, carboxylic acid derivatives, enolic acid derivatives, fenamic acid derivatives, sulphonanilides, and selective COX-2 inhibitors. “NSAID salicylates” include, but are not limited to, aspirin (acetylsalicylic acid), diflunisal, and salsalate. “NSAID p-amino phenol derivatives” include, but are not limited to, paracetamol and phenacetin. “NSAID propionic acid derivatives” include, but are not limited to, ibuprofen, naproxen, fenoprofen, ketoprofen, dexketoprofen, fluribiprofen, oxaprozin, and loxoprofen. “NSAID carboxylic acid derivatives” include, but are not limited to, indomethacin, sulindac, etodolac, ketorolac, diclofenac. NSAID carboxylic acid derivatives include NSAID acetic acid derivatives. NSAID carboxylic acid derivatives are also referred to herein as “carboxylic acid NSAIDs.” “NSAID enolic acid derivatives” include, but are not limited to, piroxicam, meloxicam, tenoxicam, droxicam, lomoxicam, and isoxicam. “NSAID fenamic acid derivatives” include, but are not limited to, mefenamic acid, meclofenamic acid, flufenamic acid, and tolfenamic acid. “NSAID sulphonanilides” include, but are not limited to, nimesulide. “NSAID selective COX-2 inhibitors” include, but are not limited to, celecoxib, rofecoxib, valdecoxib, parecoxib, lumiracoxib, etoricoxib, and firocoxib.

The term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. Specific examples of pharmaceutically acceptable carriers and excipients are provided below.

The term “pharmaceutically acceptable salts” refers to any acid or base addition salt whose counter-ions are non-toxic to the subject to which they are administered in pharmaceutical doses of the salts. Specific examples of pharmaceutically acceptable salts are provided below.

The terms “pharmaceutically effective amount”, “therapeutically effective amount” or “therapeutically effective dose” refer to the amount of a compound such as a selective bacterial β-glucuronidase inhibitor that will elicit the biological or medical response of a tissue, system, animal, or human that is being sought by the researcher, veterinarian, medical doctor or other clinician. The term “therapeutically effective amount” includes that amount of a compound such as a selective bacterial β-glucuronidase inhibitor that, when administered, is sufficient to prevent development of, or alleviate to some extent, one or more of the symptoms of the condition or disorder being treated. The therapeutically effective amount will vary depending on the compound such as a selective bacterial β-glucuronidase inhibitor, the disorder or conditions and its severity, the route of administration, time of administration, rate of excretion, drug combination, judgment of the treating physician, dosage form, and the age, weight, general health, sex and/or diet of the subject to be treated. In the context of the present method, a pharmaceutically or therapeutically effective amount or dose of a selective bacterial β-glucuronidase inhibitor includes an amount that is sufficient to prevent development of, or reduce the size or numbers of, an intestinal ulcer.

The terms “prevent”, “preventing” “prevention” and grammatical variations thereof as used herein, refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

The terms “selectively inhibit(s)”, “selective inhibitor” and the like means that a β-glucuronidase inhibitor reduces bacterial, but not mammalian, β-glucuronidase activity. In one example, the β-glucuronidase inhibitor binds to and prevents bacterial, but not mammalian, β-glucuronidases from hydrolyzing glucuronides.

The term “subject” is defined herein to include animals such as mammals, including, but not limited to, primates (e.g., humans), cows, sheep, goats, horses, dogs, cats, rabbits, rats, mice and the like. In preferred embodiments, the subject is a human.

The terms “treat”, “treating”, “treatment” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. In some instances, the terms “treat”, “treating”, “treatment” and grammatical variations thereof, include partially or completely reducing the size of an intestinal ulcer, reducing the number of intestinal ulcers, and reducing the severity of an intestinal ulcer as compared with prior to treatment of the subject or as compared with the incidence of such symptom in a general or study population. The terms “treat”, “treating”, “treatment” and grammatical variations thereof, can also include partially or completely decreasing intestinal permeability as compared with prior to treatment of the subject or as compared with the incidence of such symptom in a general or study population. The terms “treat”, “treating”, “treatment” and grammatical variations thereof, can also include partially or completely decreasing bleeding of the intestinal mucosa as compared with prior to treatment of the subject or as compared with the incidence of such symptom in a general or study population. The terms “treat”, “treating”, “treatment” and grammatical variations thereof, can also include partially or completely reducing an intestinal inflammatory response as compared with prior to treatment of the subject or as compared with the incidence of such symptom in a general or study population.

The term “ulcer” refers to a discontinuity of the epithelium or epithelial layer or a break in the epithelium or epithelial layer that stops it from continuing its normal function. The term “epithelium” includes mucous epithelium as found in the gastrointestinal tract. The term “gastrointestinal ulcer” is used to refer to an ulcer that exists in either the intestines or the stomach. “Intestinal ulcers” are of particular importance in the present invention and can arise in any portion of the intestinal tract including the small intestine (duodenum, jejunum and ileum) and the large intestine (cecum and colon). In some embodiments, the intestinal ulcers are small intestine ulcers. In further embodiments, the intestinal ulcers are ulcers of the duodenum, jejunum or ileum. In still further embodiments, the intestinal ulcers are ulcers of the jejunum and/or ileum.

### III. Methods of Treating the Adverse Intestinal Effects of NSAIDs

Methods of treating an adverse intestinal effect of a nonsteroidal anti-inflammatory drug (NSAID) in a subject by administering a therapeutically effective amount of a selective bacterial β-glucuronidase inhibitor to the subject are described herein. NSAIDs cause a multitude of adverse effects in a subject to which they are administered. These adverse effects include, but are not limited to, the formation or growth of a gastrointestinal ulcer, gastrointestinal bleeding, an increase in gastrointestinal permeability, the loss of intestinal villi, and increased gastrointestinal inflammatory responses. In some embodiments, the methods provided herein treat more than one adverse intestinal effect of an NSAID.

In certain embodiments of the method, administration of a selective bacterial β-glucuronidase inhibitor to a subject results in a reduction in the size or severity of an intestinal ulcer and/or reduces the number of intestinal ulcers as compared with prior to treatment of the subject or as compared with the incidence of such ulcer symptom in a general or study population. In other or further embodiments, administration of a selective bacterial β-glucuronidase inhibitor to a subject results in partially or completely decreasing intestinal permeability as compared with prior to treatment of the subject or as compared with the incidence of such symptom in a general or study population. In still other or further embodiments, administration of a selective bacterial β-glucuronidase inhibitor to a subject results in partially or completely reducing bleeding of the intestinal mucosa as compared with prior to treatment of the subject or as compared with the incidence of such symptom in a general or study population. In still other or further embodiments, administration of a selective bacterial β-glucuronidase inhibitor to a subject results in partially or completely reducing an intestinal inflammatory response as compared with prior to treatment of the subject or as compared with the incidence of such symptom in a general or study population.

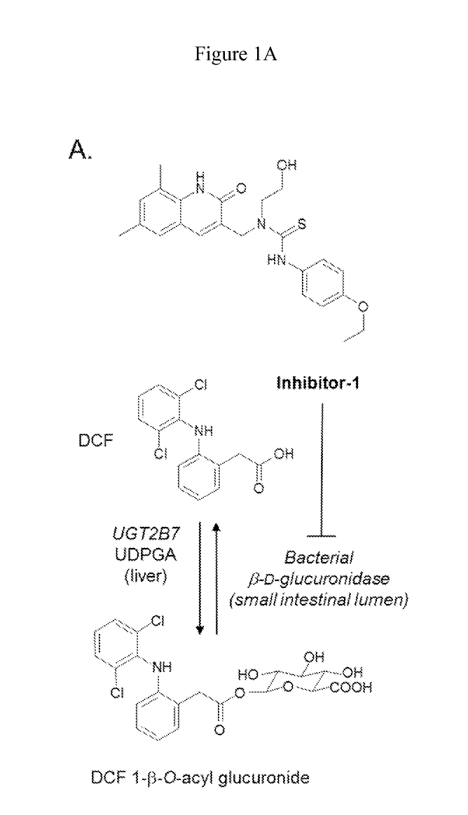

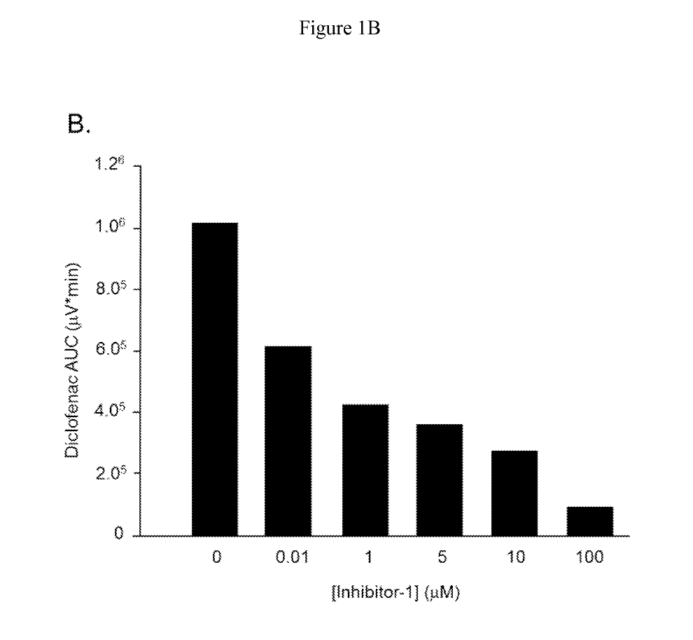

In certain embodiments, the NSAID is a carboxylic acid NSAID. Carboxylic acid NSAIDs are characterized by the inclusion of a carboxylic acid moiety (R—COOH). Carboxylic acid NSAIDs include, but are not limited to, indomethacin, sulindac, etodolac, ketorolac, and diclofenac. The examples below describe the carboxylic acid NSAID, diclofenac (or DCF). The chemical structure of diclofenac is shown in FIG. 1A. It is to be understood that all carboxylic acid NSAIDs that form glucuronides are substrates for bacterial β-glucuronidases and can therefore be targeted by the methods provided herein. Accordingly, methods of treating an adverse intestinal effect of a carboxylic acid NSAID in a subject by administering a therapeutically effective amount of a selective bacterial β-glucuronidase inhibitor to the subject are provided.

In other embodiments, the NSAID is a propionic acid NSAID. Propionic acid NSAIDs include, but are not limited to, ibuprofen, naproxen, fenoprofen, ketoprofen, dexketoprofen, fluribiprofen, oxaprozin, and loxoprofen. In one embodiment, the NSAID is ketoprofen. In still other embodiments, the NSAID is an enolic acid NSAID. Enolic acid NSAIDs include, but are not limited to, piroxicam, meloxicam, tenoxicam, droxicam, lomoxicam, and isoxicam. In further embodiments, the NSAID is a fenamic acid NSAID. Fenamic acid NSAIDs include, but are not limited to, mefenamic acid, meclofenamic acid, flufenamic acid, and tolfenamic acid. In one embodiment, the NSAID is mefanamic acid. In still further embodiments, the NSAID is a sulphonanilide NSAID. Sulphonanilide NSAIDs include, but are not limited to, nimesulide. In other embodiments, the NSAID is a selective COX-2 inhibitor NSAID. Selective COX-2 inhibitor NSAIDs include, but are not limited to, celecoxib, rofecoxib, valdecoxib, parecoxib, lumiracoxib, etoricoxib, and firocoxib. Accordingly, methods of treating an adverse intestinal effect of a propionic acid NSAID, an enolic acid NSAID, a fenamic acid NSAID, a sulphonanilide NSAID, or a selective COX-2 inhibitor NSAID in a subject by administering a therapeutically effective amount of a selective bacterial β-glucuronidase inhibitor to the subject are provided.

The selective bacterial β-glucuronidase inhibitors used in the methods provided herein include, but are not limited to, those described by Wallace et al. (Science, 330:831-835 (2010)). These glucuronidase inhibitors were found to be selective for inhibition of bacterial β-glucuronidases and do not inhibit mammalian orthologs of bacterial β-glucuronidases. It was also found that these selective bacterial β-glucuronidase inhibitors did not kill enteric bacteria or mammalian cells when used to protect mice from CPT-11 chemotherapeutic-induced toxicity. Accordingly, the selective bacterial β-glucuronidase inhibitors include compounds having one or more of the following chemical formulas:

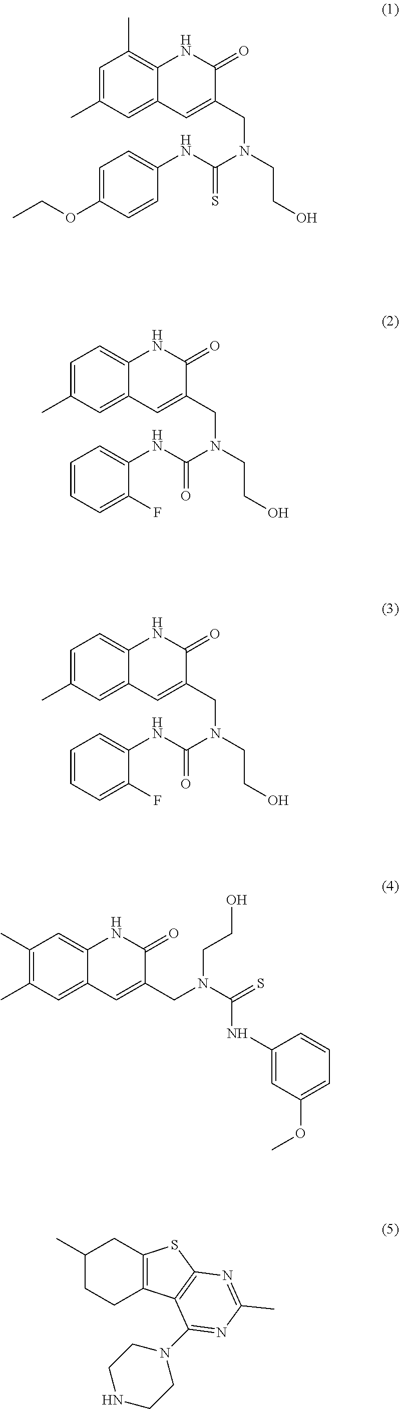

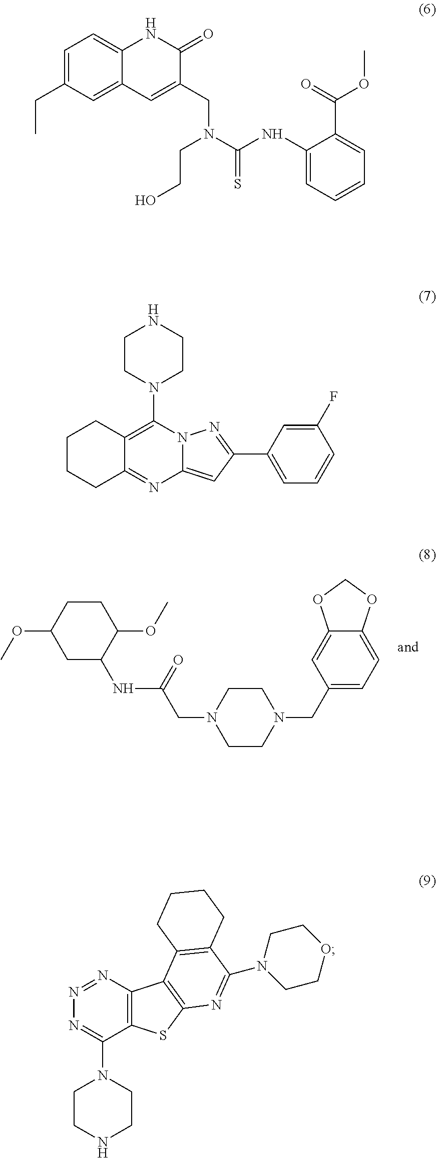

and active derivatives thereof.

The first listed selective bacterial β-glucuronidase inhibitor denoted as chemical formula “(1)” above is also referred to herein as Inh-1. Example I below demonstrates for the first time that Inh-1 is a potent inhibitor of E. coli β-D-glucuronidase diclofenac 1-β-O-acyl glucuronide (DCF-AG) cleavage. FIG. 1A schematically shows the DCF/glucuronic acid conjugation reaction, cleavage by bacterial β-glucuronidases, and inhibition by Inh-1. This discovery is important as it suggests that Inh-1 could have an effect on DCF recycling. As noted above, it is known that a portion of the hepatic diclofenac pool is conjugated in vivo with glucuronic acid to form a water-soluble 1-β-O-acyl glucuronide. This acyl glucuronide (AG) is readily excreted across the hepatocanalicular membrane via Mrp2 into the biliary tree (Seitz and Boelsterli, Gastroenterology, 115:1476-1482 (1998)) and delivered to more distal sites, i.e., the jejunum and ileum (Boelsterli and Ramirez-Alcantara, Curr. Drug Metab., 12:245 (2011)). During this transport, a portion of the AG is converted to iso-glucuronides by spontaneous acyl migration of the aglycone along the sugar ring (Dickinson and King, Life Sci., 70:25-36 (2001)). Diclofenac AG (but not the iso-glucuronides) can be cleaved by bacterial β-glucuronidase in the lumen of the small bowel. The released DCF is then taken up by enterocytes and undergoes enterohepatic circulation, thus re-exposing the mucosa repeatedly.

As further proof that Inh-1 could have an effect on DCF recycling in vivo, Example I also demonstrates that Inh-1 decreased the release of free DCF from DCF-AG in a concentration-dependent manner resulting in >90% inhibition at 100 μM inhibitor (FIG. 1B). These data demonstrate that bacterial β-D-glucuronidase is involved in the hydrolytic cleavage of DCF-AG and that this reaction can be inhibited by Inh-1 in a concentration-dependent manner.

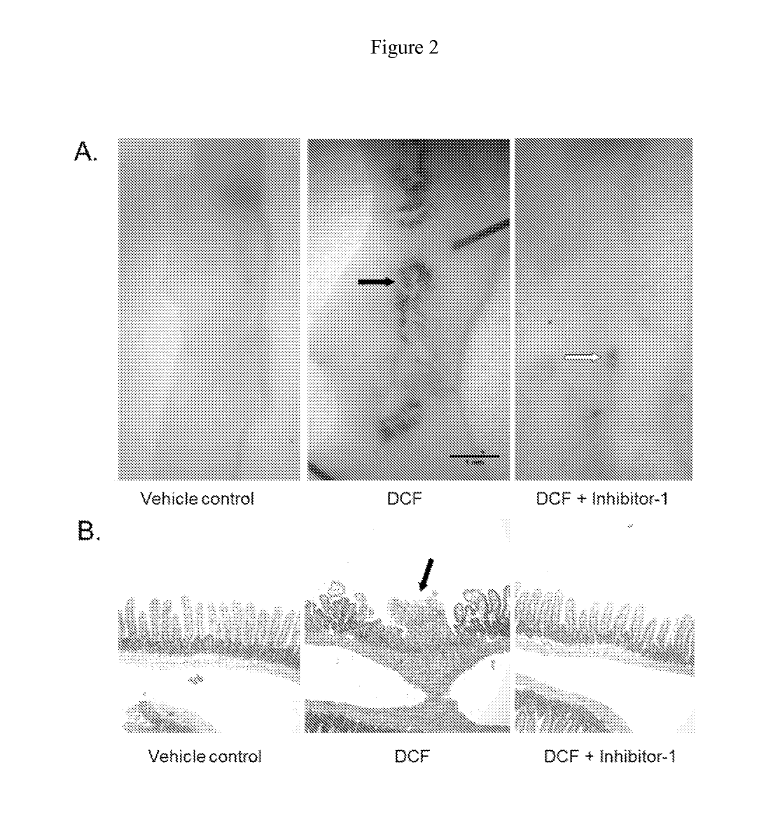

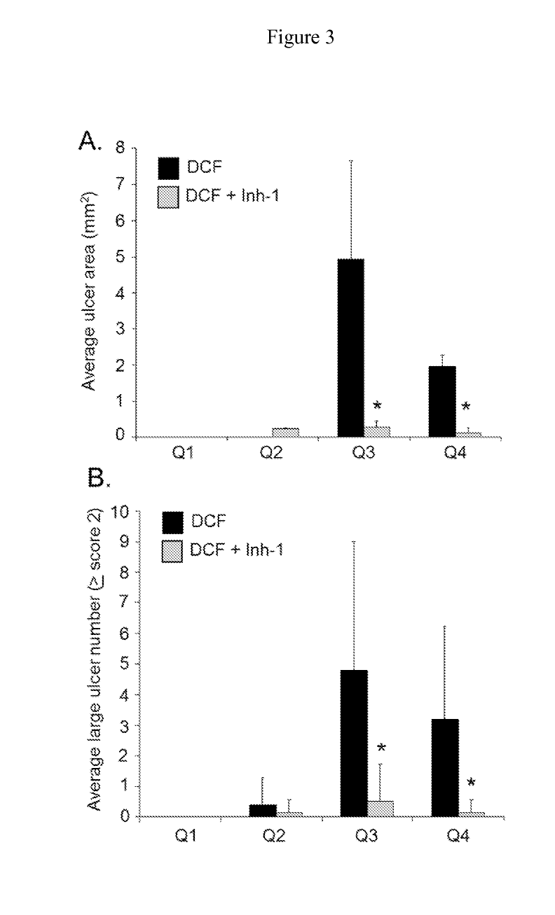

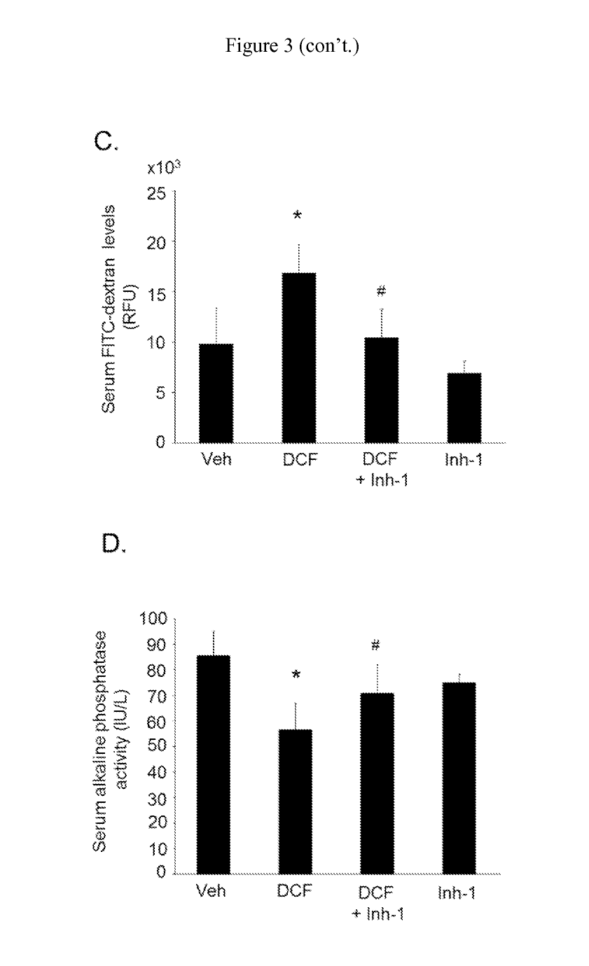

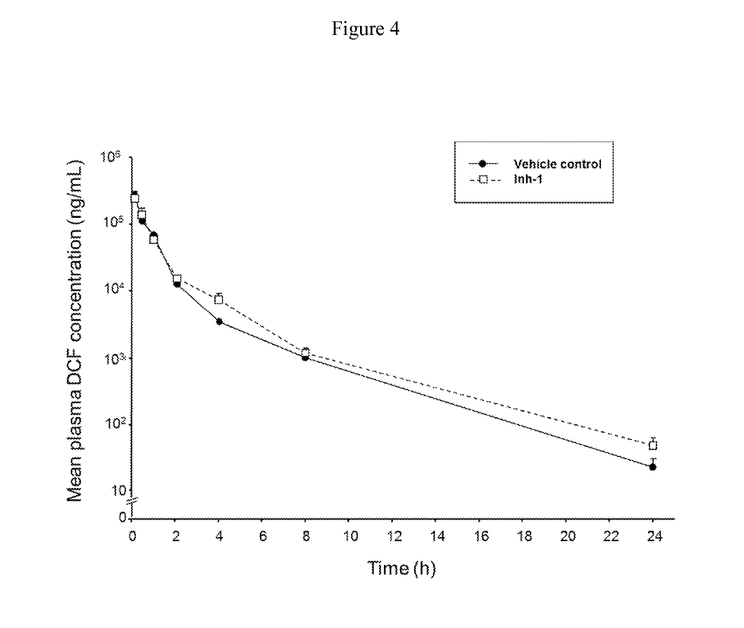

Importantly, subsequent Examples II and III confirm that Inh-1 affords significant protection from DCF-induced acute small intestinal injury in mice. The methods described herein provide such protection and in vivo effectiveness of a selective bacterial β-glucuronidase inhibitor for the first time. Histopathological analysis revealed that pretreatment with Inh-1 largely prevented the formation of DCF-induced ulcers characterized by loss of villi and evidence of an inflammatory response (FIG. 2B). Ulcers which were abundant in DCF-alone treated mice were greatly reduced both in size and number after co-treatment with Inh-1 (FIGS. 3A, B). Accordingly, in one embodiment, a method of treating a carboxylic acid NSAID-induced ulcer in a subject by administering to the subject a therapeutically effective amount of an Inh-1, or an active derivative thereof, is provided. Also provided is a method of treating a carboxylic acid NSAID-induced inflammatory response in a subject by administering to the subject a therapeutically effective amount of an Inh-1, or an active derivative thereof. In a preferred embodiment, the inflammatory response is a gastrointestinal or an intestinal inflammatory response.

FIG. 3C further demonstrates that administration of Inh-1 to a subject protects the subject from an increase in the permeability of the intestine as associated with or caused by a carboxylic acid NSAID Inh-1 also largely prevented a carboxylic acid NSAID-induced decrease in serum alkaline phosphatase (FIG. 3D). Accordingly, methods of treating a carboxylic acid NSAID-induced increase in intestinal permeability in a subject by administering to the subject a therapeutically effective amount of Inh-1, or an active derivative thereof, are provided.

The data provided herein for the first time provide novel insights into the previous uncertainties of whether acyl glucuronides (AGs), which are electrophilic species with differential reactivity towards protein targets, might be directly involved in the toxicity of carboxylic acid-containing drugs (Spahn-Langgut et al., Drug-induced Liver Disease, Kaplowitz, N and DeLeve, L eds., pp. 125-127, Informa Health Care, New York, 2007; Boelsterli and Ramirez-Alcantara, Curr. Drug Metab., 12:245-252 (2011)). If DCF-AG were indeed the toxic species, as opposed to the free parent drug or certain oxidative metabolites, then co-administration of Inh-1 would increase the degree of enteropathy rather than protect from it. Thus, the traditional concept stating that NSAID AGs are critically involved in intestinal toxicity (Seitz and Boelsterli, Gastroenterology, 115:1476-1482 (1998)) can now be clarified to the following: glucuronide metabolites remain critical, but they may merely be a transport form delivering the drug from the liver to more distal sites in the gastrointestinal tract, i.e., the jejunum and ileum (Boelsterli and Ramirez-Alcantara, Curr. Drug Metab., 12:245-252 (2011)).

This revised concept may also provide a possible explanation as to why rodents are extremely sensitive to NSAID enteropathy, which may be related to the route of NSAID metabolite excretion. Rodents, especially rats, secrete higher levels of glucuronides into bile, due to the lower molecular weight cutoff (300-400 Da) at the canalicular plasma membrane (Klaassen and Watkins, Pharmacol. Rev., 36:1-67 (1984)), and DCF glucuronides are clearly above this threshold. For example, DCF administration to gallbladder-cannulated mice revealed that the amount of biliary DCF-AG was 117-fold higher than that of the parent DCF, and 22-fold higher than that of ring-hydroxylated DCF (Lagas et al., Mol. Pharmacol., 77:687-694 (2010)). In contrast, the canalicular export cutoff in humans is higher (approximately 500 Da) (Bailey and Dickinson, Chem. Res. Toxicol., 9:659-666 (1996)), and a larger portion of glucuronides are excreted renally rather than via the hepatobiliary route. Nevertheless, a considerable amount of DCF is excreted via bile in humans; initial studies had estimated this to be 10-20% (Stierlin et al., Xenobiotica, 9:601-610 (1979)), but subsequent studies had revealed that approximately 75% of total DCF clearance was in the form of glucuronides including AGs of the major oxidative metabolite, 4′-hydroxy-DCF (Kumar et al., J. Pharmacol. Exp. Ther., 303:969-978 (2002)).

If such a large proportion of an administered dose of DCF is excreted via the hepatobiliary route—42% of [¹⁴C]-DCF equivalents appeared in bile during 60 minutes after intravenous administration of 5 mg/kg in mice (Lagas et al., Mol. Pharmacol., 77:687-694 (2010))—then the question may arise why inhibition of glucuronide cleavage by bacterial β-glucuronidase did not have any apparent effects on the DCF plasma level versus time curve (FIG. 4, Table 1). A plausible reason may be that in mice the amount of DCF-AG in bile was only approximately 4% of the dose; the remaining conjugated metabolites (approximately 30% of the dose) were either iso-glucuronides (which are not a substrate for β-glucuronidase), glucuronides of the 4′-hydroxy or 5′-hydroxy metabolites (Lagas et al., Mol. Pharmacol., 77:687-694 (2010)), or even taurine conjugates (Sarda et al., Xenobiotica, in press, 2011). Thus, despite the inhibition of the bacterial enzyme, the therapeutic efficacy of the NSAID likely remains intact.

Accordingly, the methods described herein provide an important and novel treatment of adverse intestinal effects of NSAIDs in vivo, particularly methods of administering a selective bacterial β-glucuronidase inhibitor or a pharmaceutically acceptable salt, ester or pro-drug thereof, in a pharmaceutically acceptable carrier or diluent, for the treatment of an adverse intestinal effect of a carboxylic acid NSAID, a propionic acid NSAID, an enolic acid NSAID, a fenamic acid NSAID, a sulphonanilide NSAID or a selective COX-2 inhibitor NSAID. It will be appreciated that the selective bacterial β-glucuronidase inhibitors described herein may be derivatized at functional groups to provide pro-drug derivatives that are capable of conversion back to the parent compounds in vivo. Examples of such pro-drugs include the physiologically acceptable and metabolically labile ester derivatives, such as methoxymethyl esters, methoyltiomethyl esters, or pivaloyloxymethyl esters derived from a hydroxyl group of a selective bacterial β-glucuronidase inhibitor or a carbamoyl moiety derived from an amino group of a selective bacterial β-glucuronidase inhibitor. Additionally, any physiologically acceptable equivalents of the selective bacterial β-glucuronidase inhibitors, similar to metabolically labile esters or carbamates, which are capable of producing the selective bacterial β-glucuronidase inhibitors in vivo, are within the scope of the methods provided herein.

If pharmaceutically acceptable salts of the selective bacterial β-glucuronidase inhibitors are utilized for administration, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexamoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, priopionate, succinate, tartrate, thiocynate, tosylate, undecanoate, hydrohalides, sulfates, phosphates, nitrates, sulphamates, malonates, salicylates, methylene-bix-b-hydroxynaphthoates, gentisates, isethionates, di-p-toluoyltartrates, ethanesulphonates, cyclohexylsulphamates, quinates, and the like. Pharmaceutically base addition salts include, but are not limited to, those derived from alkali or alkaline earth metal bases or conventional organic bases, such as tri-ethylamine, pyridine, piperidine, morpholine, N-methylmorpholine, ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, etc. Furthermore, the basic nitrogen-containing groups may be quaternized with agents such as lower alkyl halides, such as methyl, ethyl, propyl and butyl chlorides, bromides and iodides, dialkyl sulfates, such as dimethyl, diethyl, dibutyl and diamyl sulfates, long chain halides, such as decyl, lauryl, myristyl and stearyl chlorides, bromides and iodides, aralkyl halides, such as benzyl and phenethyl bromides and others. Water or oil-soluble or dispersible products are thereby obtained.

The compounds utilized in the methods provided herein may also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The selective bacterial β-glucuronidase inhibitors can be manufactured by methods well known in the art such as conventional granulating, mixing, dissolving, encapsulating, lyophilizing, or emulsifying processes, among others. Compositions containing the selective bacterial β-glucuronidase inhibitors may be produced in various forms including granules, precipitates, particulates, powders, tablets, capsules, syrup, suppositories, injections, emulsions, elixirs, suspensions or solutions. These compositions may optionally contain stabilizers, pH modifiers, surfactants, bioavailability modifiers and combinations of these.

Pharmaceutically acceptable carriers that may be administered with the selective bacterial β-glucuronidase inhibitors include ion exchangers, alumina, aluminum stearate, lecithin, serum proteins, such as human serum albumin, buffer substances, such as phosphates, glycine, sorbic acid, potassium sorbate, partial glyceride mixtures of vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The selective bacterial β-glucuronidase inhibitors may additionally be administered in compositions that include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents.

Administration of a selective bacterial β-glucuronidase inhibitor with one or more suitable pharmaceutical excipients as advantageous are carried out via any of the accepted modes of administration and as the term “administering” is defined above. Preferably, the selective bacterial β-glucuronidase inhibitors are administered orally. The selective bacterial β-glucuronidase inhibitor can be administered once or repeatedly, e.g. at least 2, 3, 4, 5, 6, 7, 8, or more times, or by continuous infusion. Suitable sites of administration include, but are not limited to, mouth, gastrointestinal, skin, bronchial, anal, vaginal, eye and ear.

The selective bacterial β-glucuronidase inhibitors are administered before, concurrently, or after administration of an NSAID. In one embodiment, the selective bacterial β-glucuronidase inhibitor is administered concurrently with the NSAID. In another embodiment, the selective bacterial β-glucuronidase inhibitor is administered before administration of an NSAID. When administered before an NSAID, the selective bacterial β-glucuronidase inhibitor is preferably administered 1, 2, 3, 4, 5, 6, 8, 10, 12, 18, 24, 30, or 45 minutes, hours, or days prior to the NSAID administration.

### EXAMPLES

The methods provided herein will be further understood by reference to the following non-limiting examples.

### Example I

### Inh-1 is a Potent Inhibitor of E. coli β-D-Glucuronidase Diclofenac 1-β-O-Acyl Glucuronide Cleavage

It was first determined that diclofenac-aglycone (DCF-AG) is a substrate for bacterial β-glucuronidase in vitro by demonstrating that purified E. coli β-glucuronidase converted DCF-AG (various concentrations) to its aglycone in vitro (data not shown). Expression and purification of E. coli β-glucuronidase was conducted as previously described in Wallace et al., 2010. Diclofenac was obtained from Sigma (St. Louis, Mo.). Diclofenac-1-β-O-acyl glucuronide was obtained from LC Scientific, Inc. (Ontario, Canada). All chemicals were of the highest grade available.

To assess the inhibition characteristics of Inh-1, increasing concentrations of Inh-1 were added to the incubation system containing 4 mM DCF-AG Inh-1 ((6,8-dimethyl-2-oxo-1,2-dihydroquinolin-3-yl)methyl)-3-(4-ethoxyphenyl)-1-(2-hydroxyethyl)thiourea) as described in Wallace et al., 2010 was synthesized in house. DCF-AG assays were performed at 50 μL total volume in 96-well assay plates (Costar). Reactions consisted of the following:

twenty-five microliters Assay Buffer (2% DMSO, 100 mM NaCl, and 100 mM HEPES, pH 6.8), 15 μL substrate (DCF-AG), 5 μL of Inh-1 solution, and 5 μL of 5 nM enzyme. Each reaction was quenched with trichloroacetic acid (TCA) to a final concentration of 10% TCA. Samples were centrifuged at 13,000×g for 10 minutes to pellet the precipitate prior to sample detection. HPLC-UV detection of the DCF product was carried out in a similar protocol as previously reported (Seitz et. al., Chem. Res. Toxicol., 11:513-519 (1998)) using a Phenomenex Luna 5 μm C18(2) reverse-phased HPLC column The AUC for the peak corresponding to the product DCF was calculated for each inhibitor concentration.

Inh-1 decreased the release of free DCF in a concentration-dependent manner resulting in greater than 90% inhibition at 100 μM inhibitor (FIG. 1B). The IC₅₀ was calculated to be approximately 140 nM. These data demonstrate that bacterial β-D-glucuronidase is involved in the hydrolytic cleavage of DCF-AG and that this reaction can be inhibited by Inh-1 in a concentration-dependent manner.

### Example II

### Inh-1 Protects Mice from Diclofenac-Induced Small Intestinal Ulceration

To determine the toxicologic consequences of inhibiting intestinal bacterial β-glucuronidase, mice were treated with a single ulcerogenic dose of DCF (60 mg/kg, intraperitoneal) with or without pretreatment with Inh-1 (10 μg/mouse, b.i.d., ×2 days) and the extent of drug-induced enteropathy was analyzed. Male C57BL/6J mice were obtained from The Jackson Laboratory (Bar Harbor, Me.). The mice were acclimatized for three weeks before the experiments and were 10-12 weeks of age at the start of the experiments. The animals were kept on a 14:10-hour light:dark cycle. They received mouse chow (Teklad Global Rodent Diet, Harlan Laboratories, Boston, Mass.) and water ad libitum. All studies were approved by the Institutional Animal Care and Use Committee of the University of Connecticut. Diclofenac was dissolved in 10% (in PBS) Solutol HS-15 solution and administered intraperitoneally (60 mg/kg) in a volume of 10 μL/g body weight. Previous studies in rats had revealed that the extent of small intestinal injury was qualitatively and quantitatively similar for both oral or intraperitoneal routes of administration as the development of enteropathy critically depends on portal delivery of DCF to the liver followed by hepatobiliary export of DCF conjugates (Seitz and Boelsterli, Gastroenterology, 115:1476-1482 (1998)). All animals were treated at five hours before the start of the dark cycle. Inh-1 or vehicle (0.5% methyl cellulose) was administered by oral gavage b.i.d. (10 μg/mouse), starting one day before DCF administration and with the last dose given one hour before DCF to minimize drug-drug interactions. Control animals received methyl cellulose and/or Solutol HS-15.

As expected, mice receiving DCF alone developed typical nitroblue tetrazolium-(NBT-) positive areas of ulceration in the jejunum and ileum (FIG. 2A), while vehicle controls did not exhibit any apparent pathological lesions. In contrast, in mice pretreated with Inh-1, DCF caused only few isolated and small NBT-positive areas. Histopathological analysis of these lesions revealed that DCF induced typical ulcers characterized by loss of villi and an inflammatory response, involving the entire mucosa (FIG. 2B), while pretreatment with Inh-1 largely prevented these changes. Quantitative analysis revealed that the ulcers, which were abundant in DCF-alone treated mice in quartiles 3 and 4, were greatly reduced both in size and number after co-treatment with Inh-1 (FIGS. 3A, B).

Similarly, the DCF-associated decrease in serum alkaline phosphatase (ALP) activity (an established biomarker of enteropathy) (Ramirez-Alcantara et al., Am. J. Physiol.(Gastrointest. Liver Physiol.), 297:G990-G998 (2009)) was largely prevented by Inh-1 pretreatment (FIG. 3D). Enteropathy was assessed and graded as described previously (Ramirez-Alcantara et al., Am. J. Physiol. (Gastrointest. Liver Physiol.), 297:G990-G998 (2009)). Briefly, mice were sacrificed by CO₂ inhalation at 18 hours post-DCF (when the development of mucosal injury was maximal). A midline incision was made and blood was obtained via cardiac puncture. Serum was prepared and frozen at −80° C. until use for analysis. The entire small intestine (from the gastroduodenal junction to the ileocecal junction) was removed and opened longitudinally along the anti-mesenteric side. The tissue was rinsed in cold PBS and incubated for 15 minutes in 1 mM nitroblue tetrazolium (NBT) solution containing 16 mM HEPES-NaOH, 125 mM NaCl buffer, 3.5 mM KCl, and 10 mM glucose. Next, the tissues were fixed in 10% zinc formalin for 24 hours, washed, and transferred to 70% ethanol. The intestine was metrically divided into four quartiles of equal length and evaluated at 10× magnification for quantitative and qualitative analysis of mucosal injury, and the lesions were assigned to the respective quartiles. The following scoring system was used: 0, no apparent lesions; 1, small erosions or ulcers (<0 1 mm); 2, medium ulcers (0.1-0.8 mm); 3, large confluent ulcers (>0.8 mm) In addition, the total area of the lesions was quantified by Image J software. The formalin-fixed tissue was embedded in paraffin, and 5-μm sections were stained with hematoxylin/eosin for histopathological evaluation. Serum activity of alkaline phosphatase (ALP) was measured with a kinetic colorimetric kit (BioAssay Systems, Hayward, Calif.).

Furthermore, the transmucosal permeability of FITC-dextran (a high-molecular weight, non-metabolizable branched glucan labeled with fluorescein, which normally is poorly absorbed into the systemic circulation) was increased by almost two-fold after DCF alone but remained at vehicle control levels following pretreatment with Inh-1 (FIG. 3C). Intestinal permeability changes were determined as described (Napolitano et al., Shock, 5:202-207 (1996)) with minor modifications. In brief, mice were administered FITC-dextran (4 kDa) by oral gavage (400 mg/kg, in 0.5% methyl cellulose) 3 hours prior to blood collection by cardiac puncture. Serum was prepared and stored at −80° C. until used. After dilution of the serum (1:10), fluorescence was recorded in black 96-well plates at λ=490 nm/530 nm (excitation/emission, respectively). The fluorescence measurements were linear with respect to the concentration range, and the absolute values were determined with a standard curve. All data were expressed as mean±SD. If there was normal distribution, a standard ANOVA was used followed by Dunnett's test for multiple comparisons versus the control group. When normality failed, a Kruskal-Wallis one way ANOVA on ranks was used followed by Dunn's test for multiple comparison versus the control group. A P value of ≦0.05 was considered statistically significant.

Taken together, these data indicate that Inh-1 afforded significant protection from DCF-induced acute small intestinal injury in mice.

### Example III

### Pharmacokinetic Profiling of Diclofenac in Mice

To assess whether inhibition of intestinal bacterial hydrolysis of DCF glucuronides would alter the disposition of DCF, and thus potentially impair its pharmacologic efficacy at the target tissues, a pharmacokinetic study was conducted in mice following a single administration of DCF (60 mg/kg) with and without pretreatment with Inh-1 (FIG. 4). Mice were administered Inh-1 (10 μg/mouse, bid, ×2 days; n=9) or vehicle (0.5% methyl cellulose; n=9), followed by DCF (60 mg/kg, intraperitoneal). From each mouse, maximally three blood samples were serially collected by retro-orbital puncture, so that for each time point samples were collected from three mice. Blood samples (approximately 120 μl) were obtained at 0.16, 0.5, 1, 2, 4, 8, and 24 hours post-dose Immediately after collection, plasma was prepared and stored below −70° C. until analysis. All samples were processed for analysis by precipitation using acetonitrile and analyzed by LC-MS/MS (Sai Advantium Pharma Ltd., Pune, India), with a lower limit of quantitation for diclofenac of 5.0 ng/ml. Briefly, an ACE 3 C₁₈ column (150×4.6 mm; 3.5 μm) was used; the mobile phase for gradient elution consisted of 5 mM ammonium formate in water and acetonitrile. Mass spectrometry was performed with an API 4000 equipped with a turbo ion spray source. Non-compartmental-Analysis module in WinNonlin® (version 5.2) was used to assess the pharmacokinetic parameters.

Analysis of the DCF plasma level-over-time curve revealed that there was a typical bi-exponential decrease in plasma concentration with an apparent Tₘₐₓ of 10 minutes and an apparent Cₘₐₓ in the high micromolar range (see Table 1 below). Pretreatment with Inh-1 did not significantly change these parameters, and the overall systemic exposure to DCF (AUC₀₋₂₄ₕ) was not significantly different from that in DCF alone-treated mice (exposure ratio vehicle group/Inh-1 group=0.99). These data indicate that the amount of DCF acyl glucuronide (and possibly other glucuronide metabolites) excreted into the bile and reaching the lower GI is sufficient to trigger mucosal injury to the small intestine, but not high enough to significantly alter the systemic exposure if its hydrolytic cleavage is prevented by the bacterial β-glucuronidase inhibitor.

TABLE 1
Effects of Inh-1 on the pharmacokinetic parameters of diclofenac

| | Tₘₐₓ | Cₘₐₓ | AUC_{0-24 h} | T_{1/2} |
|---|---|---|---|---|
| Treatment | (h) | (ng/mL) | (ng/mL * h) | (h) |
| Diclofenac | 0.16 | 264,868 ± | 208,425 | 2.82 |
| | | 46,216 | | |
| Diclofenac + | 0.16 | 240,115 ± | 210,014 | 2.77 |
| Inh-1 | | 18,020 | | |

Male C57BL/6 mice were pretreated with Inh-1 (10 μg/mouse, p.o.) or vehicle (0.5% methyl cellulose) b.i.d. for 2 consecutive days. Diclofenac (60 mg/kg, intraperitoneal) was administered one hour after the last dose of Inh-1. Blood was drawn after 0.16, 0.5, 1, 2, 4, 8, and 24 hours post-diclofenac, and plasma drug concentrations were determined by LC-MS/MS. The data are mean±SD (n=3 mice for each time point; maximally three serial sampling times per mouse).

It is understood that the examples and embodiments described herein are for illustrative purposes only and that various modifications or changes in light thereof will be suggested to person skilled in the art and are to be included with the spirit and purview of this applications and scope of the appended claims. All publications, patents and patent applications cited herein are hereby incorporated by reference in their entirety for all purposes.

What is claimed is:

1. A method of treating one or more adverse intestinal effects of a nonsteroidal anti-inflammatory drug (NSAID) in a subject comprising administering to the subject a therapeutically effective amount of a bacterial β-glucuronidase inhibitor.

2. The method of claim 1, wherein the one or more adverse intestinal effects comprise the formation or growth of an intestinal ulcer, increased intestinal permeability, the loss of intestinal villi, bleeding of the small intestinal mucosa, or an increased intestinal inflammatory response.

3. The method of claim 1, wherein the one or more adverse intestinal effects comprise the formation or growth of an intestinal ulcer.

4. The method of claim 1, wherein the NSAID is selected from the group consisting of: propionic acid derivatives, acetic acid derivatives, enolic acid derivatives, fenamic acid derivatives, sulphonanilides, and selective COX-2 inhibitors.

5. The method of claim 4, wherein the NSAID is a carboxylic acid derivative.

6. The method of claim 5, wherein the carboxylic acid derivative is selected from the group consisting of indomethacin, sulindac, etodolac, ketorolac, and diclofenac.

7. The method of claim 5, wherein the carboxylic acid derivative is diclofenac.

8. The method of claim 1, wherein the selective bacterial β-glucuronidase inhibitor is selected from the group consisting of: 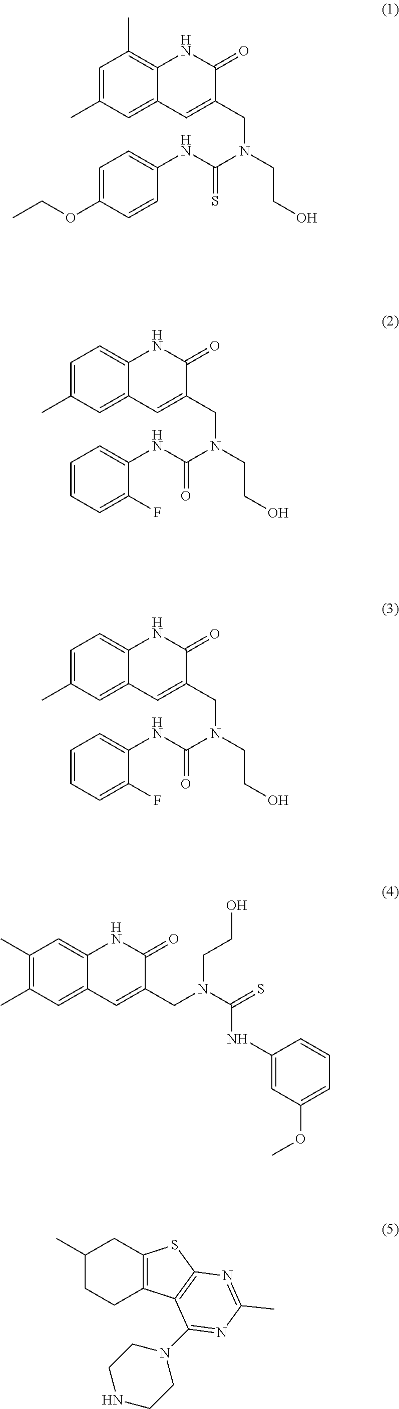 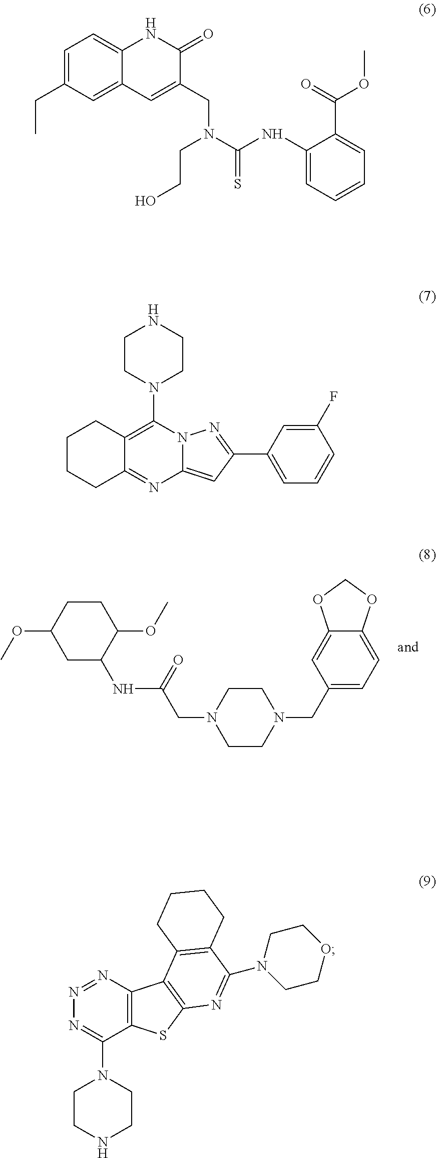
and active derivatives thereof.

9. The method of claim 1, wherein the selective bacterial β-glucuronidase inhibitor is a compound having the chemical structure: 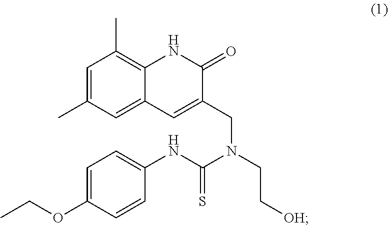 [SMILES: CCOc1ccc(NC(=S)N(CCO)Cc2cc3cc(C)cc(C)c3[nH]c2=O)cc1]
or an active derivative thereof.

10. The method of claim 1, wherein the bacterial β-glucuronidase inhibitor is administered to the subject before administration of the NSAID.

11. he method of claim 1, wherein the bacterial β-glucuronidase inhibitor is administered to the subject concurrently with administration of the NSAID.

12. The method of claim 1, wherein the bacterial β-glucuronidase inhibitor is administered to the subject after administration of the NSAID.

13. The method of claim 1, wherein the bacterial β-glucuronidase inhibitor is administered orally.
